(19) United States
(12) Patent Application Publication
(10) Pub. No.: US 2014/0061116 A1
(43) Pub. Date: Mar. 6, 2014

(54) EXCHANGER DEVICE

(71) Applicants: Thomas Schmitz-Rode, Aachen (DE); Ulrich Steinseifer, Hauset (BE); Jutta Arens, Aachen (DE); Peter Schlanstein, Aachen (DE); Ralf Borchardt, Aachen (DE)

(72) Inventors: Thomas Schmitz-Rode, Aachen (DE); Ulrich Steinseifer, Hauset (BE); Jutta Arens, Aachen (DE); Peter Schlanstein, Aachen (DE); Ralf Borchardt, Aachen (DE)

(73) Assignee: Dritte Patentportfolio Beteiligungsgesellschaft mbH & Co. KG, Schoenefeld (DE)

(21) Appl. No.: 14/115,221

(22) PCT Filed: May 2, 2012
(86) PCT No.: PCT/EP2012/057971

§ 371 Date: Nov. 1, 2013

(30) Foreign Application Priority Data
May 4, 2011 (DE) 10 2011 100 439.8

(51) Int. Cl. A61M 1/16

(52) U.S. Cl. CPC A61M 1/1698
USPC 210/321.87

(57) ABSTRACT

An exchanger device for transferring mass and/or energy between a first medium and a second medium includes a chamber having a first inlet and first outlet of the first medium and through which the first medium can flow. The chamber is equipped with at least one, preferably a plurality, of one mass- and/or energy-permeable exchanger hollow fibers connected at one end to a second inlet and at the other end to a second outlet of the second medium. The second medium can flow through the fiber(s) and the first medium can flow around the fiber(s). The chamber is equipped with at least one pump element for displacing the first medium in and out of the chamber in a pulsing manner. The pump element has an elastically deformable element, is connected to a third inlet of a third medium used as a driving medium, and is expandable by the third medium.

### CROSS-REFERENCE TO RELATED APPLICATIONS

This application is a Section 371 of International Application No. PCT/EP2012/057971, filed May 2, 2012, which was published in the German language on Nov. 8, 2012, under International Publication No. WO 2012/150233 A1 and the disclosure of which is incorporated herein by reference.

### BACKGROUND OF THE INVENTION

The invention relates to an exchanger device for effectuating a mass and/or energy exchange between a first medium and a second medium, the device having a chamber comprising a first inlet and a first outlet of the first medium and through which the first medium can flow, in which chamber at least one mass-permeable and/or energy-permeable exchanger hollow fiber, preferably a plurality of mass-permeable and/or energy-permeable exchanger hollow fibers, is/are disposed which can be connected at a first end to a second inlet and at the other end to a second outlet of the second medium and through which the second medium can flow and around which the first medium can flow, wherein at least one pumping element is arranged within the chamber by which the first medium can be displaced from the chamber and sucked into the chamber in a pulsating manner, and which exhibits an elastically deformable element.

Generic devices of this type are used for instance in medical technology, and there in particular in applications for blood purification such as dialysis, blood separation or else as artificial lungs (oxygenators).

In the field of application of oxygenators it is thereby provided for the blood as a first medium to be allowed to flow through a chamber in which at least one mass-permeable and/or energy-permeable hollow fiber, in a preferred embodiment a plurality of mass-permeable and/or energy-permeable hollow fibers, is/are arranged, through which the second medium, here in particular oxygen, can flow and around which the first medium flows. Hollow fiber modules for this purpose are described in International Patent Application Publication No. WO 2011/023605 A1.

When blood originating from a living being's body and enriched with CO₂ is pumped through the chamber, a mass transfer comes about on both sides of the mass-permeable and/or energy-permeable hollow fiber due to the differing partial pressures of oxygen and CO₂, in the sense that CO₂ is removed from the blood and the same is enriched with oxygen from the hollow fibers. Thus, such a device can work as an artificial lung and for instance assume the pulmonary function of a patient partially or else completely.

Insofar as an oxygenator, i.e. an artificial lung, is described in more detail in this specification, this should not be understood as a restriction but only as an exemplary application. The device described here can basically be used for the mass or energy transfer between arbitrary media, and this not only in medical technology but also in other industrial applications.

Usually, external pumps are employed to achieve adequate and defined flow rates of a first medium through the afore-mentioned chamber, in particular of the blood through the chamber. In the field of application of oxygenators this means that, in addition to the generic device, a pump needs to be provided by which blood can be pumped from a patient's body through the device and then back into the patient's body.

The cited principle requires a significant volume to arise in the generic devices and the externally used pump which has to be filled with the first medium, such as blood in this application.

An exchanger device of the afore-mentioned kind is described in International Patent Application Publication No. WO2008/104353 A1, which has an integrated pumping function and therefore does not require any external pump volumes.

### BRIEF SUMMARY OF THE INVENTION

The invention is based on the object to provide a further improved device of this type which yields advantages in particular with respect to operating and patient safety.

This object is achieved by an exchanger device of the type described at the outset for effectuating a mass and/or energy exchange between a first medium and a second medium, the device having a chamber comprising a first inlet and a first outlet of the first medium and through which the first medium can flow, in which chamber at least one mass-permeable and/or energy-permeable exchanger hollow fiber, preferably a plurality of mass-permeable and/or energy-permeable exchanger hollow fibers, is/are disposed, which can be connected at one end to a second inlet and at the other end to a second outlet of the second medium and through which the second medium can flow and around which the first medium can flow, wherein at least one pumping element is arranged within the chamber by which the first medium can be displaced from the chamber and sucked into the chamber in a pulsating manner, and which exhibits an elastically deformable element and is connected to a third inlet of a third medium serving as a driving medium, and is expandable by the third medium, characterized in that the first inlet and the first outlet for the first medium are each provided with a controllable valve for interrupting the inflow or outflow of the first medium in a time-controlled manner, and a flow sensor is provided on the first inlet and first outlet which is in communication with a sensor signal input of a pump and valve control unit and/or a heat source in the circulation of the third medium, and/or a bubble detector is provided on the first outlet which is in communication with a sensor signal input of the pump and valve control unit.

Appropriate further developments of the inventive idea are the object of the depending claims and described below.

According to the invention, utilizing one of the two media participating in the mass or energy exchange at the same time as the medium for reducing the pumping function is abandoned, and this function is realized with an additional (third) medium. Therefore, the idea to provide at least one separate inlet for that medium serving as a driving medium for the pumping function is at the same time a part of the invention.

In an embodiment of the invention, a plurality of elastically deformable pumping hollow fibers is disposed within the chamber as pumping elements, and these are in communication with the third inlet.

In the embodiment of the device as an oxygenator, particularly important in medical use, the second medium is oxygen, and correspondingly an oxygen storage or an oxygen source is connected to the second inlet of the device. Air can be used in a particularly simple and cost-efficient manner as the third medium, with an air pump arranged for pulsating operation then being connected to the corresponding (third) inlet. The pulsating expansion and contraction of the pumping element or especially the plurality of elastically expandable hollow fibers essential to the pumping function can hereby be advantageously achieved with a unilaterally closed pumping element or unilaterally sealed hollow fibers; thus, there is no outlet for the third medium in this embodiment.

In one configuration it is provided for the chamber to comprise a first sub-chamber adjacent the third inlet, on the one hand, and adjacent the entry of the pumping hollow fiber or the entries of the pumping hollow fibers, on the other, a second sub-chamber adjacent the second inlet, and a third sub-chamber adjacent the first inlet and outlet and receiving the exchanger hollow fiber or exchanger hollow fibers. This subdivision of the device interior is for the purpose of a clear functional allocation and delimitation, especially with a reasonable distribution of the media across the available inner volume of the device for guaranteeing an optimized pumping and exchanging function.

In another implementation, a fluid, in particular physiological saline solution, serves as the third medium, and a corresponding fluid reservoir is provided. In one configuration of this implementation, an energy exchange function is linked to the mass transfer function in order to keep a patient's blood to be enriched with oxygen at the same time at body temperature. In this configuration, the pumping element or the pumping hollow fibers are open on both sides and integrated into a fluid circulation of the third medium, which has a heat source associated for heating the third medium.

A practical functional subdivision of the exchanger device provides in this case for the chamber to comprise a first sub-chamber adjacent the third inlet, on the one hand, and adjacent the entry of the pumping hollow fiber or the entries of the pumping hollow fibers, on the other, a second sub-chamber adjacent the second inlet, a third sub-chamber adjacent the first inlet and outlet and receiving the exchanger hollow fiber or exchanger hollow fibers as well as a fourth sub-chamber adjacent the outlet of the pumping hollow fiber or the outlets of the pumping hollow fibers, on the one hand, and adjacent the third outlet, on the other.

A further implementation of the invention provides for the first inlet and outlet for the first medium to be provided each with a controllable valve for interrupting the inflow or outflow of the second medium in a time-controlled manner. In an especially advantageous manner which avoids providing a closing body in the supply or discharge line, the controllable valves in this case are preferably realized as hose pinch valves. The device comprises in particular a pump and valve control unit designed for the synchronized control of the pump for the third medium and the valves at the first inlet and outlet for causing the first medium to be conveyed through the chamber from the inlet to the outlet.

A further implementation is characterized by a flow sensor at the first inlet and/or outlet which is in particular in communication with a sensor signal input of the pump and valve control and/or the heat source in the circulation of the third medium. A further implementation, which may be advantageously combined with the latter, comprises a bubble detector at the first outlet which is in particular in communication with a sensor signal input of the pump and valve control unit. Such sensors allow precision of the control as well as patient safety of the proposed device to be further improved.

In a further configuration of the invention, the exchanger device has a cylindrical or prismatic housing, with the first and second inlet being in particular disposed in a circumferential wall and the third inlet in a front surface, and the or each exchanger hollow fiber being arranged essentially perpendicular to the cylinder axis or longitudinal extension of the prism, and the or each pumping element being oriented essentially parallel to the cylinder axis or longitudinal extension of the prism. In one configuration of this housing construction, the first outlet is disposed in the circumferential wall of the housing, in particular opposite the first inlet, and the second outlet is disposed in the front surface opposite the connection for the third medium or close to this, offset with respect to the first inlet and the first outlet in the circumferential wall.

In an important implementation or use, the inventive exchanger device is a blood oxygenator. A further important implementation or use is one as a dialysis machine. Principally, the device in both applications can at least in part be realized to be implantable.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. Advantages and expediency of the invention will incidentally result from the following description of implementation examples and aspects on the basis of the Figures. In the drawings:

FIG. 1 is a schematic flow diagram of an arrangement according to one embodiment of the invention, in which an inventive exchanger device is used in an important medical application;

FIG. 2 is a schematic cross-sectional representation of an embodiment of the inventive device; and

FIG. 3 is a schematic cross-sectional representation of a further embodiment including the identification of various functional areas.

### DETAILED DESCRIPTION OF THE INVENTION

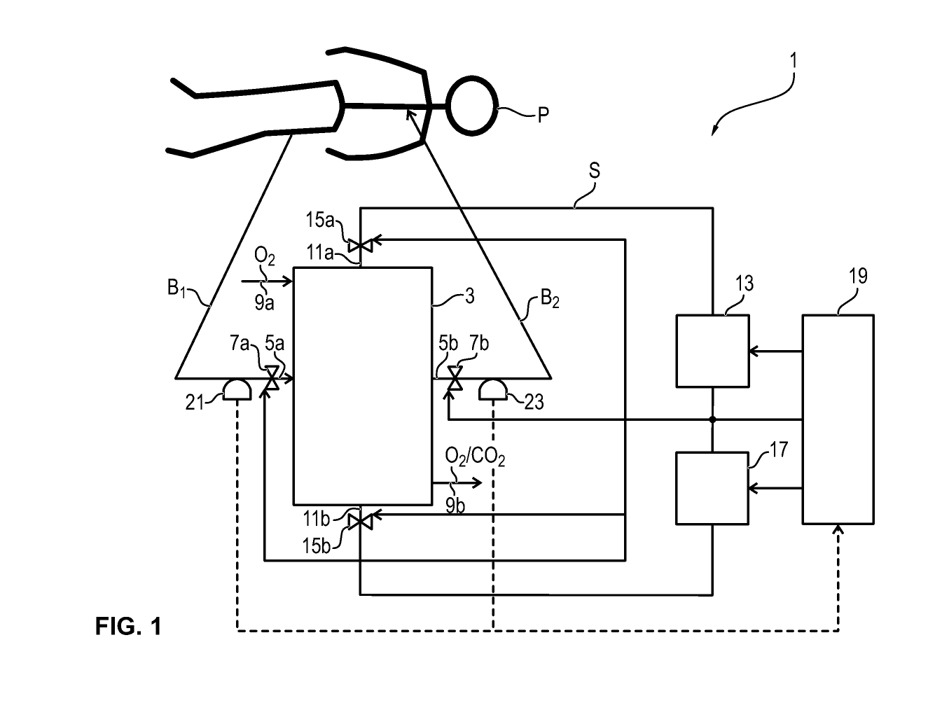

The oxygenator arrangement 1 shown in FIG. 1 for realizing an “artificial lung” on a patient P comprises as a key element an exchanger device (oxygenator) 3 to which oxygen-deficient blood B₁ from the patient P is supplied via hose lines (not particularly designated), and from which oxygen-rich blood B₂ is discharged and supplied again to the patient. For supplying the blood B₁, the exchanger device 3 has a first inlet 5a, and for discharging the oxygen-enriched blood B₂ it has a first outlet 5b. A first controllable valve 7a is disposed on the first inlet 5a, and a second controllable valve 7b is disposed on the outlet 5b.

Oxygen O₂ is supplied to the exchanger device 3 via a second inlet 9a, and an oxygen/carbon dioxide mixture O₂/CO₂ is discharged from the device via a second outlet 9b. In the embodiment shown, the exchanger device 3 is operated with physiological saline solution S as the driving medium, which is supplied to the device via a third inlet 11a and discharged from the device via a third outlet 11b. The saline solution S is guided through the exchanger device 3 in a pulsating manner into a fluid circulation by a suitable fluid pump 13, for which purpose a controllable valve 15a or 15b is also provided respectively on the third inlet 11a and the third outlet 11b. The saline solution can be appropriately temperature-adjusted by a heating device 17 in order to compensate for heat loss of the blood B₂ during the oxygen enrichment via an additional heat exchanger function of the exchanger device 3.

For controlling the operation of the feed pump 13, the heating device 17 and the controllable valves 7a, 7b, 15a and 15b, a pump and valve control unit 19 having an integrated heating control function is provided which has usual input and programming means (not illustrated). On the input side, the pump and valve control unit 19 incidentally is in communication with a blood flow sensor 21 on the first inlet 5a and a bubble detector 23 on the first outlet 5b and implemented for processing sensor signals from these sensors 21, 23 for appropriately controlling the pump 13 and the controllable valves in order to adjust an adequate flow rate and avoid bubbles in the oxygen-enriched blood B₂. In a modified embodiment (not illustrated), a sampling and/or a non-invasive sensor-based detection of the blood oxygen saturation may be provided on each of the blood inlet and outlet and also the signals of corresponding sensors or analysis results respectively may be evaluated within the pump and valve control unit for defining an appropriate control regime of the arrangement 1.

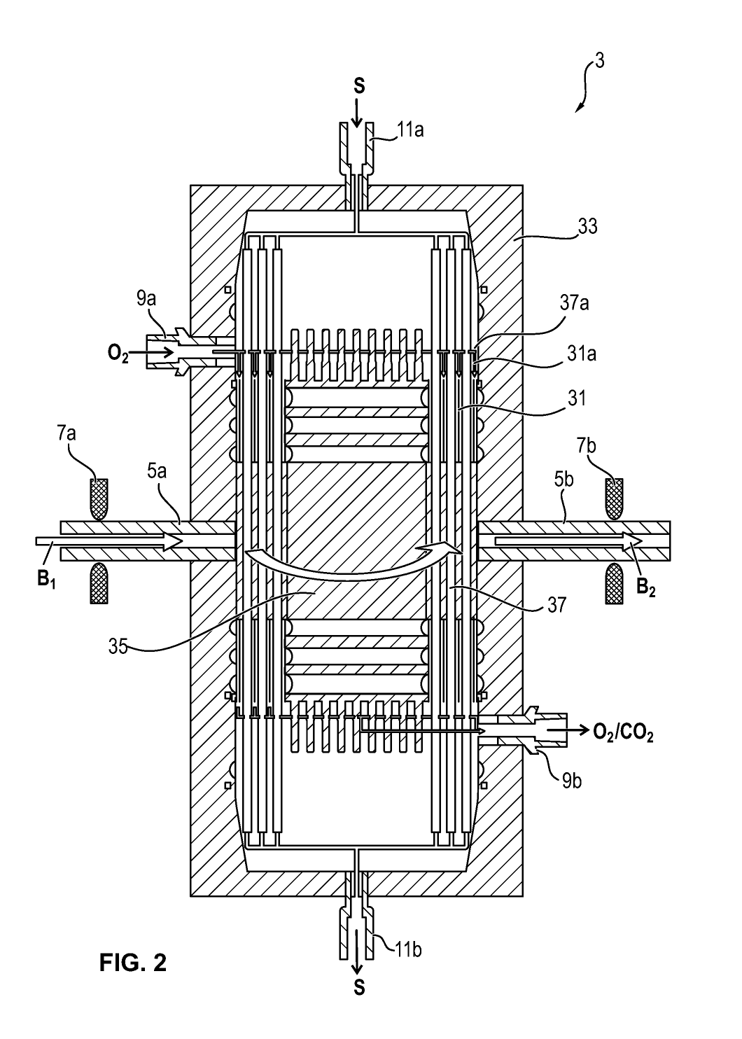

FIG. 2 shows the inner structure of the exchanger device 3 in a schematic cross-sectional representation. Parts shown in FIG. 1 and already described above are designated by the same numerals and are not explained again here. The embodiment differs from that shown in FIG. 1 in that on each of the third inlet and outlet 11a, 11b controllable valves are not present or shown.

The functional key element of the exchanger device 3 is a bundle of gas-permeable hollow fibers 31 which are disposed in a cylindrical housing 33 of the exchanger device oriented essentially in the direction of the cylinder axis and the one ends of which are adjacent the second inlet (the oxygen supply) 9a, and the other ends of which are adjacent the second outlet (the oxygen/carbon dioxide outlet) 9b. Into these hollow fibers 31, oxygen is introduced, and the oxygen flows through the fibers to the opposite end. In the central area of the interior (the “chamber”) 35 of the exchanger device 3, the blood supplied through the first inlet 5a flows around the hollow fibers 31, and an enrichment in oxygen and at the same time a depletion in carbon dioxide takes place in the blood; the hollow fibers 31 thus act as exchanger hollow fibers.

For conveying the media to be set into a gas exchange, actually the blood B₁/B₂ (also referred to above as the first medium) and the oxygen O₂ (also referred to above as the second medium), a pulsating pumping movement is realized within the exchanger device 3, and namely by a pulsating feeding of the saline solution S into a group of pumping hollow fibers (silicone hoses) 37 arranged essentially along the cylinder axis and which are elastically expandable. The pumping hollow fibers 37 are in particular of a spiral configuration, wherein fibers having a diameter of 2 mm and a wall thickness of 0.15 mm can be employed and spiraled using (temporarily introduced) A1 cores. The pulse-like expansion thereof as a consequence of the pulsating feeding of the saline solution S as the driving medium causes the desired conveyance processes at the blood inlet and outlet 5a, 5b in conjunction with a correspondingly controlled actuation of the valves 7a, 7b. The groups of hollow fibers 31 and 37, respectively, are connected to the corresponding inlets and outlets 9a, 9b and 11a, 11b via appropriate connectors 31a and 37a, respectively.

If in deviation from the illustrated version, pressurized air is used as the driving medium of the exchanger device, a recycling can be dispensed with and the third outlet (11b in FIG. 2) omitted, wherein modified pumping hollow fibers sealed at their ends facing away from the inlet of the third medium will then be used. In such a version, the second outlet (for the consumed exchange gases) can then also be placed on the lower front surface of the exchanger device housing.

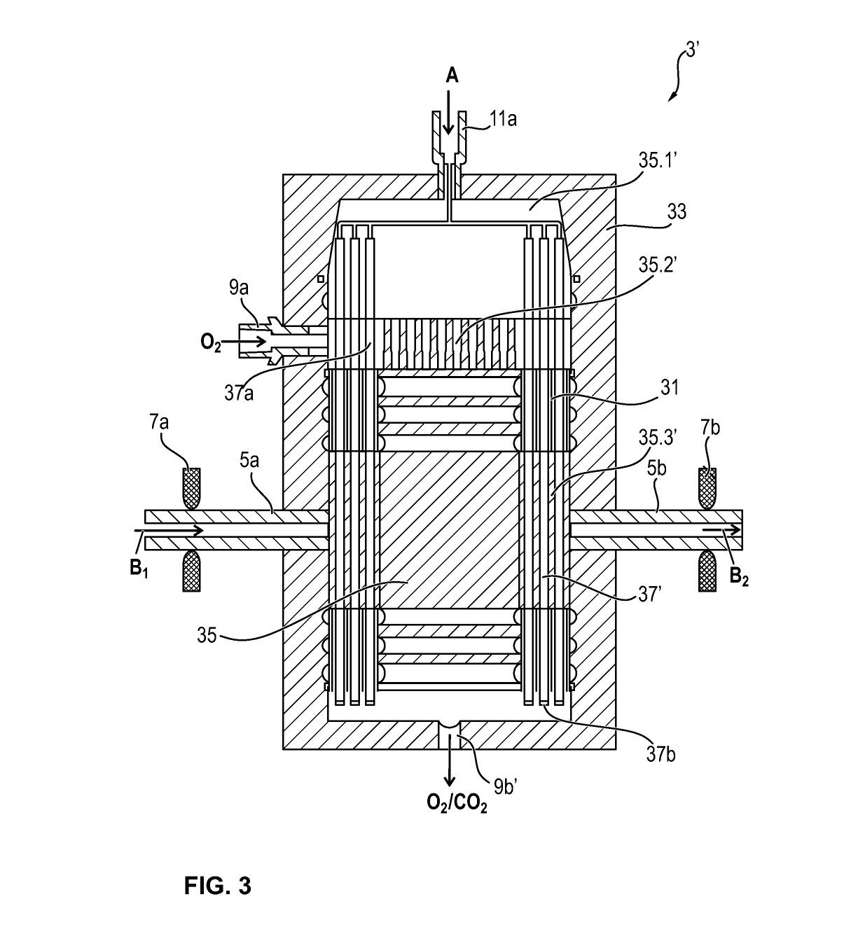

Such a modified exchanger device 3′ is shown in FIG. 3—likewise in a schematic cross-sectional representation. Here as well, the mode of designation follows the principle of FIGS. 1 and 2, and parts or areas already described above will not be explained again. The cited sealing of the ends of the pumping hollow fibers 37′ facing away from the third inlet 11a for the air A serving in this case as the driving medium, is realized here by end plugs 37b. The sealing of the fiber ends may be performed for instance by a commercial two-component silicone using a centrifuge. It can be recognized that the third outlet has been omitted and the second outlet 9b′ positioned in its place.

As the essential functional areas in the exchanger device 3′ interior, a first sub-chamber 35.1′ adjacent the third inlet 11a, a second sub-chamber 35.2′ adjacent the second inlet 9a and lastly a third sub-chamber 35.3′ adjacent the first inlet and outlet 5a, 5b can be distinguished. In the first sub-chamber, the driving medium (here the air A) is fed and “buffered,” in the second sub-chamber 35.2′, the oxygen feeding and distributing takes place, and in the third sub-chamber 35.3′ the conveying of the blood through the exchanger device finally takes place in conjunction with the functionally relevant gas exchange and a heat exchange as need be (as described above in the context of FIG. 2). The elastically expandable pumping hollow fibers or at least essential portions thereof are also in the third sub-chamber.

The described device operates with a continuous oxygen supply via the second inlet 9b and air pressure pulses fed via the third inlet 11a, which can be fed in an appropriate implementation at between 20 and 140 pulses/min and a pressure differential of up to 600 mm Hg, wherein the relationship between the systolic and diastolic phase can be varied between 0.2 and 0.8; all of these being merely useful exemplary values. The air pressure pulses cause a periodic expansion and contraction of the pumping hollow fibers and a conveying of the blood through the third sub-chamber 35.3′ in combination with a temporally appropriately controlled opening and closing of the blood inlet and outlet 5a, 5b via the valves 7a, 7b disposed there, in conjunction with the desired oxygen enrichment through the oxygen present in the exchanger hollow fibers 31 surrounded by the blood.

The implementation of the invention is not limited to the examples described and aspects emphasized here but it is also possible in numerous modifications which reside within the framework of skilled action.

1-18. (canceled)

19. An exchanger device for effectuating a mass and/or energy exchange between a first medium and a second medium, the device comprising:
a chamber having a first inlet and a first outlet for the first medium and adapted for flow of the first medium therethrough,
at least one mass-permeable and/or energy-permeable exchanger hollow fiber disposed in the chamber, the at least one exchanger hollow fiber being connected at one end to a second inlet and at another end to a second outlet for the second medium, the at least one hollow fiber being adapted for flow of the second medium therethrough and for flow of the first medium therearound,
at least one pumping element arranged within the chamber and adapted for displacing the first medium into and out of the chamber in a pulsating manner, the at least one pumping element comprising an elastically deformable element,
the at least one pumping element being connected to a third inlet for a third medium serving as a driving medium, wherein the at least one pumping element is expandable by the third medium,
the first inlet and the first outlet each having a controllable valve for interrupting inflow or outflow of the first medium in a time-controlled manner, and
a flow sensor provided on the first inlet and outlet and being in communication with a sensor signal input of a pump and valve control unit and/or a heat source in a circulation of the third medium, and/or a bubble detector provided on the first outlet and being in communication with the sensor signal input of the pump and valve control unit.

20. The exchanger device according to claim 19, wherein the chamber has a plurality of the mass-permeable and/or energy-permeable exchanger hollow fibers disposed therein.

21. The exchanger device according to claim 19, wherein the chamber has a plurality of elastically deformable pumping hollow fibers disposed therein as pumping elements and in communication with the third inlet.

22. The exchanger device according to claim 19, further comprising an oxygen storage connected to the second inlet as a source of the second medium, and an air pump arranged for pulsating operation connected to the third inlet as a source of the third medium.

23. The exchanger device according to claim 21, wherein the chamber has a first sub-chamber adjacent the third inlet and adjacent entries of the pumping hollow fibers, a second sub-chamber adjacent the second inlet, and a third sub-chamber adjacent the first inlet and outlet and receiving the at least one exchanger hollow fiber.

24. The exchanger device according to claim 21, wherein ends of the pumping hollow fibers facing away from the third inlet are sealed.

25. The exchanger device according to claim 19, further comprising an oxygen storage connected to the second inlet as a source of the second medium, and a fluid pump arranged for pulsating operation and in communication with a fluid reservoir connected to the third inlet as a source of the third medium.

26. The exchanger device according to claim 25, wherein the chamber has a third outlet, the pumping hollow fibers are open on both sides and integrated into a fluid circulation of the third medium, which has a heat source associated for heating the third medium.

27. The exchanger device according to claim 26, wherein saline solution is contained in the fluid reservoir and optionally in the fluid circulation.

28. The exchanger device according to claim 25, wherein the chamber has a first sub-chamber adjacent the third inlet and adjacent entries of the pumping hollow fibers, a second sub-chamber adjacent the second inlet, a third sub-chamber adjacent the first inlet and outlet and receiving the at least one exchanger hollow fiber, and a fourth sub-chamber adjacent the outlets of the pumping hollow fibers and adjacent the third outlet.

29. The exchanger device according to claim 19, wherein the controllable valves comprise hose pinch valves.

30. The exchanger device according to claim 19, wherein the pump and valve control unit is designed for synchronized control of the pump for the third medium and the controllable valves at the first inlet and the first outlet for causing the first medium to be conveyed through the chamber from the first inlet to the first outlet.

31. The exchanger device according to claim 19, the device having a cylindrical or prismatic housing, wherein the first inlet and the second inlet are disposed in a circumferential wall of the housing and the third inlet is disposed in a front surface of the housing, the at least one exchanger hollow fiber being arranged essentially perpendicular to an axis of the cylindrical housing or a longitudinal extension of the prismatic housing, and the at least one pumping element being oriented essentially parallel to the axis or the longitudinal extension.

32. The exchanger device according to claim 31, wherein the first outlet is disposed in the circumferential wall of the housing opposite the first inlet, and the second outlet is disposed in the front surface opposite a connection for the third medium or adjacent thereto offset with respect to the first inlet and the first outlet in the circumferential wall.

33. The exchanger device according to claim 19, wherein the device is configured as a blood oxygenator.

34. The exchanger device according to claim 19, wherein the device is configured as a dialysis machine.
